(19) United States
(12) Patent Application Publication
(10) Pub. No.: US 2017/0022144 A1
(43) Pub. Date: Jan. 26, 2017

(54) COSMETIC COMPOSITIONS COMPRISING ESTERS BASED ON 2-PROPYLHEPTANOIC ACID

(71) Applicant: Cognis IP Management GmbH, Dusseldorf (DE)

(72) Inventor: Markus Dierker, Duesseldorf (DE)

(21) Appl. No.: 15/284,009

(22) Filed: Oct. 3, 2016

Related U.S. Application Data
(63) Continuation of application No. 12/599,173, filed on Nov. 6, 2009, now abandoned.

(30) Foreign Application Priority Data
May 7, 2007 (EP) 07009138.4

(51) Int. Cl. C07C 69/24; A61Q 19/00; A61K 8/37

(52) U.S. Cl. CPC C07C 69/24; A61K 8/37; A61Q 19/00

(57) ABSTRACT

The invention relates to esters of 2-propylheptanoic acid and also to the use of esters of 2-propylheptanoic acid in cosmetic and/or pharmaceutical preparations. The compounds are characterized by the particularly lightweight sensation thereof.

### FIELD OF THE INVENTION

The invention relates to esters of 2-propylheptanoic acid and to their use in cosmetic and/or pharmaceutical preparations, and methods for their preparation.

### PRIOR ART

In the field of cosmetic emulsions for skincare and hair care, a large number of requirements are imposed by the consumer: apart from the cleaning and care effects, which determine the intended use, value is also placed on such different parameters as highest possible dermatological compatibility, good refatting properties, elegant appearance, optimum sensory impression and storage stability.

Besides a series of surface-active substances, preparations which are used for the cleaning and care of the human skin and hair generally comprise in particular oil bodies and water. The oil bodies/emollients used are, for example, hydrocarbons, ester oils and vegetable and animal oils/fats/waxes. In order to fulfill the high requirements of the market with regard to sensory properties and optimum dermatological compatibility, new oil bodies and emulsifier mixtures are being continually developed and tested. The use of ester oils in cosmetics has been known for a long time. On account of their importance, new methods for their preparation are also being continually developed. In particular branched ester oils convey a “lighter” skin feel and are therefore intensively investigated. The use of 2-methyl-1,3-propanediol monoesters is, for example, the subject matter of DE 101 60 681, the use of 2-methyl-1,3-propanediol diesters is described in DE 101 60 682.

It was an object of the present invention to provide novel ester oils that are preferably liquid at 20° C. for cosmetic and/or pharmaceutical applications which have an improved profile in respect of the sensory properties (lightness, “nongreasy skin feel”, softness, spreadability, absorption, distributability, oiliness) and can be incorporated into a large number of cosmetic formulations. In this connection, the hydrolysis stability of the esters and the formulatability of the esters at a low pH were also of interest. Furthermore, the esters should be able to be incorporated both into W/O and also into O/W formulations. Furthermore, the esters should in particular be compatible with crystalline UV filters, pigments, antiperspirant salts and silicones. Furthermore, the esters should be oxidation-stable. Surprisingly, it has been found that esters of 2-propylheptanoic acid lead to sensorially light products. DE 103 05 562 describes ester mixtures of linear carboxylic acids with alcohols which can be used as polymer additives. WO 2006/097235 describes esters of 2-propylheptanol with linear or branched carboxylic acids, i.e. the esters of 2-propylheptanol with 2-propylheptanoic acid. G. Schomberg, Journal of Chromatography, 1964, pp. 157-177, describes on page 165, table II, entry 6, methyl 2-propylheptanoate, referred to there as methyl ester of nonanecarboxylic acid-(4). JP 10-289441 (Sony Corp.) describes on page 4, section 29, table 1, entry 2, nonadecyl 2-propylheptanoate. JP 57 006790 (Canon KK) describes 2-hexyldecyl 2-propylheptanoate on page 486, No. 11. None of the three last-mentioned documents describes cosmetic and/or pharmaceutical preparations or the use of said compounds in cosmetic and/or pharmaceutical preparations. The object of the present invention was to provide esters that are improved compared with the prior art.

### DESCRIPTION OF THE INVENTION

The invention provides esters of the general formula (I)

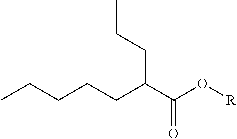

where R is an alkyl radical having 1 to 36 carbon atoms, where R may be substituted by up to 3 OH groups, with the exception of 2-propylheptyl 2-propylheptanoate, methyl 2-propylheptanoate, nonadecyl 2-propylheptanoate and 2-hexyldecyl 2-propylheptanoate.

Particular preference is given to esters of the general formula (I)

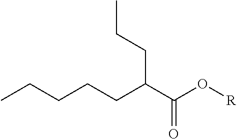

where R is an alkyl radical having 1 to 36 carbon atoms, where R can be substituted by up to 3 OH groups, with the exception of 2-propylheptyl 2-propyl-heptanoate, 2-ethylbutyl 2-propylheptanoate, 2-butyl-octyl 2-propylheptanoate, methyl 2-propylheptanoate, nonadecyl 2-propylheptanoate and 2-hexyldecyl 2-propyl-heptanoate.

Surprisingly, esters of 2-propylheptanoic acid are particularly suitable for cosmetic formulations, in particular for formulations where a “light” skin feel is important. These esters can be incorporated very readily into various formulations. Depending on chain length, branching and number of double bonds, liquid substance mixtures are obtained which are accordingly suitable as oil bodies or consistency regulators, which give bodies in emulsions. The esters exhibit high stability towards oxidation. According to the invention, a single 2-propylheptanoic acid ester can be used, or any desired mixture of different esters.

A preferred embodiment of the invention relates to esters whose total carbon number is greater than or equal to 14, in particular greater than 14, in particular greater than or equal to 16, in particular greater than 16.

A preferred embodiment of the invention relates to esters whose total carbon number is less than or equal to 24, in particular less than 24, in particular less than or equal to 22, in particular less than 22.

The alkyl radical R in formula (I) can be a saturated or unsaturated, linear or branched, aliphatic or aromatic radical. In one embodiment of the invention, it is substituted by up to 3 OH groups, in particular by up to 2 OH groups, preferably by one OH group. In one embodiment, R is an alkyl radical having 1 to 24, in particular 2 to 20, preferably 6 to 18, carbon atoms, in particular 6 to 12 carbon atoms.

Radicals R which may be mentioned are alkyl groups with a carbon number from 1 to 8, such as, for example, methyl, ethyl, propyl, isopropyl [=1-methylethyl], propenyl, isobutyl [2-methylpropyl], sec-butyl [=1-methylpropyl], tert-butyl [1,1-dimethylethyl], but-2-enyl, but-3-enyl, but-l-enyl, n-pentyl, 1-methylbutyl, 2-methylbutyl, 3-methylbutyl, 1-ethyl-propyl, 1,1-dimethylpropyl, 1,2-dimethylpropyl, 2,2-dimethylpropyl, 1-pentenyl, 2-pentenyl, 3-pentenyl, 4-pentenyl, hexyl, 1-methylpentyl, 2-methylpentyl, 3-methylpentyl, 4-methylpentyl, 1-ethylbutyl, 2-ethyl-butyl, 3-ethylbutyl, 1-hexenyl, 2-hexenyl, 3-hexenyl, 4-hexenyl, 5-hexenyl, heptyl, 1-methylhexyl, 2-methyl-hexyl, 3-methylhexyl, 4-methylhexyl, 5-methylhexyl, 1-heptenyl, 2-heptenyl, 3-heptenyl, 4-heptenyl, 5-heptenyl, 6-heptenyl, n-octyl, 2-ethylhexyl, 1,1,3,3-tetramethylbutyl.

Radicals R which may be mentioned are alkyl groups with a carbon number from 9 to 22, such as, for example, nonyl, isononyl, decyl, isodecyl, undecyl, undecenyl, dodecyl, tridecyl, isotridecyl, tetradecyl, pentadecyl, hexadecyl, octadecyl, octadecenyl, eicosanoyl, behenyl radical.

The radical R can be aromatic radicals, such as phenyl, methyl-substituted phenyl, such as, for example, tolyl, hydroxy-substituted phenyl, such as, for example, 2-hydroxyphenyl, 3-hydroxyphenyl, benzyl, methyl-substituted benzyl, hydroxy-substituted benzyl, such as, for example, 2-hydroxybenzyl or 3-hydroxybenzyl.

The radical R can be substituted by up to 3 OH groups, in one preferred embodiment, the radical R is an alkyl radical with one OH group, such as, for example, hydroxyhexyl, hydroxyoctyl, hydroxydecyl, hydroxy-dodecyl, 2,3-dihydroxy-n-propyl (from glycerol), 2-hydroxyethyl (ethylene glycol), 3-hydroxypropyl, 2-hydroxypropyl (from propylene glycol), 2-hydroxy-propyl, 3-hydroxypropyl, 4-hydroxypropyl, 1-methyl-2-hydroxypropyl, 6-hydroxyhexyl, 8-hydroxyoctyl, 10-hydroxydecyl, 12-hydroxydodecyl, 14-hydroxy-tetradecyl, 2-ethyl-2-hydroxymethyl-3-hydroxypropyl (from trimethylolpropane), 2,2-bis(hydroxymethyl)-3-hydroxypropyl (from pentaerythritol).

Likewise suitable as radical R is an alkyl radical having 36 carbon atoms which can be prepared by reacting 2-propylheptanoic acid with a C36-dimerdiol. C36-Dimerdiol refers to alpha, omega-C36-diols which can be prepared by dimerization of oleyl alcohol (=octadec-9-en-1-ol) or by hydrogenation of dimer fatty acid (esters).

Particular preference is given to esters selected from the group consisting of n-hexyl 2-propylheptanoate, n-heptyl 2-propylheptanoate, n-octyl 2-propyl-heptanoate, n-nonyl 2-propylheptanoate, n-decyl 2-propylheptanoate, n-undecyl 2-propylheptanoate, n-dodecyl 2-propylheptanoate.

The invention includes both individual esters and also mixtures of different esters.

The invention further provides the use of esters of the general formula (I),

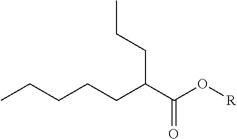

where R is an alkyl radical having 1 to 36 carbon atoms, where R can be substituted by up to 3 OH groups, with the exception of 2-propylheptyl 2-propyl-heptanoate, in cosmetic and/or pharmaceutical preparations. Particular preference is given to the use of these esters with the exception of 2-ethylbutyl 2-propylheptanoate and 2-butyloctyl 2-propylheptanoate in cosmetic and/or pharmaceutical preparations. Particular preference is given to the use of the esters according to claim 1, in particular of the esters according to claim 1 with the exception of 2-ethylbutyl 2-propylheptanoate and 2-butyloctyl 2-propylheptanoate, in cosmetic and/or pharmaceutical preparations. The esters are suitable in particular as oil bodies in cosmetic and/or pharmaceutical preparations.

The invention further provides the use of esters of the general formula (I),

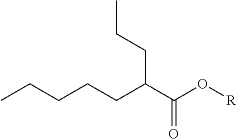

where R is an alkyl radical having 1 to 36 carbon atoms, where R can be substituted by up to 3 OH groups, with the exception of 2-propylheptyl 2-propyl-heptanoate, for preparing cosmetic and/or pharmaceutical preparations. Particular preference is given to the use of these esters, with the exception of 2-ethylbutyl 2-propylheptanoate and 2-butyloctyl 2-propylheptanoate, for preparing cosmetic and/or pharmaceutical preparations. Particular preference is given to the use of the esters according to claim 1, in particular of the esters according to claim 1 with the exception of 2-ethylbutyl 2-propylheptanoate and 2-butyloctyl 2-propylheptanoate, for preparing cosmetic and/or pharmaceutical preparations.

The invention further provides in particular the use of esters of the general formula (I),

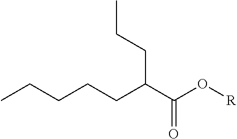

where R is an alkyl radical having 1 to 36 carbon atoms, where R can be substituted by up to 3 OH groups, with the exception of 2-propylheptyl 2-propyl-heptanoate, in cosmetic and/or pharmaceutical preparations for the wetting or impregnation or coating of utility and/or hygiene wipes which are used for cleaning the body and/or for body care. Particular preference is given to this use of these esters with the exception of 2-ethylbutyl 2-propylheptanoate and 2-butyloctyl 2-propylheptanoate. Particular preference is given to this use of the esters according to claim 1, in particular of the esters according to claim 1 with the exception of 2-ethylbutyl 2-propylheptanoate and 2-butyloctyl 2-propylheptanoate.

Also provided is a method for preparing the esters according to the invention, where a mixture comprising 2-propylheptanoic acid and the corresponding alcohol is reacted.

The method according to the invention likewise covers the preparation of ester mixtures in which 2-propylheptanoic acid is reacted together with the corresponding alcohol mixtures.

In one preferred embodiment of the invention, the mixture comprising 2-propylheptanoic acid and the corresponding alcohol is reacted with the addition of an esterification catalyst.

In one preferred embodiment, the mixture comprising 2-propylheptanoic acid and the corresponding alcohol is heated, the water which forms is continuously removed and the crude product is then distilled. The method can be carried out with the addition of an esterification catalyst, e.g. with acidic catalysis or basic catalysis. In one preferred embodiment, the method is carried out without the addition of solvents, preferably with starting materials which are as anhydrous as possible. In one preferred embodiment of the method, a tin catalyst is used. Suitable tin catalysts are, for example, tin oxalate (e.g. Fascat® 2001), tin oxide (SnO, Fascat 2000) and tinIV catalysts such as dibutyltin diacetate (Fascat® 4200), dibutyltin oxide (Fascat® 4201), and dibutyltin laurate (Fascat® 4202) or tin oxide (SnO), which were formerly marketed by Atofina and are currently marketed by Arkema.

Preferably, the esterification is carried out at temperatures between 100-300° C., in particular 200-250° C.

In a further embodiment, the catalyst used is at least one enzyme. Suitable enzymes are all enzymes or enzyme mixtures known to the person skilled in the art which are able to catalyze the esterification of alcohol and acid, examples which may be mentioned being lipases, acyl transferases and esterases. The enzymatically catalyzed esterification is usually carried out at temperatures of 20 to 100° C., preferably 40 to 80° C.

The esters according to the invention can also be prepared by reacting an alkyl ester of 2-propylheptanoic acid (e.g. methyl ester or butyl ester) and the corresponding alcohol with the addition of a transesterification catalyst. This method too is suitable for preparing ester mixtures.

In one such method, the mixture comprising the alkyl ester of 2-propylheptanoic acid and the corresponding alcohol is heated with the addition of the esterification catalyst, the water which forms is continuously removed and the crude product is then distilled. In one preferred embodiment, the method is carried out without the addition of solvents, preferably with starting materials which are as anhydrous as possible. Preferably, the esterification is carried out at temperatures between 100-300° C., in particular 200-250° C. Transesterification catalysts which can be used are all transesterification catalysts known to the person skilled in the art, preference being given to using sodium methylate or tetraalkyl titanate as transesterification catalyst.

As catalyst, it is also possible to use at least one enzyme. Suitable enzymes are all enzymes or enzyme mixtures known to the person skilled in the art which are able to catalyze the transesterification of alcohol and acid methyl ester, examples which may be mentioned being lipases, acyl transferases and esterases. The enzymatically catalyzed esterification is usually carried out at temperatures of from 20 to 100° C., preferably 40 to 80° C.

Cosmetic/pharmaceutical preparations

The esters according to the invention permit the preparation of stable cosmetic and pharmaceutical emulsions with a particularly light skin feel.

The present invention therefore further provides cosmetic and/or pharmaceutical preparations comprising (a) at least one ester of the general formula (I),

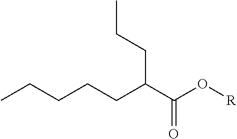

where R is an alkyl radical having 1 to 36 carbon atoms, where R can be substituted by up to 3 OH groups, with the exception of 2-propylheptyl 2-propylheptanoate, (b) at least one emulsifier (b-1) and/or surfactant (b-2) and/or wax component (b-3) and/or polymer (b-4) and/or a further oil body (b-5).

Preference is given to cosmetic and/or pharmaceutical preparations comprising
(a) at least one ester of the general formula (I),

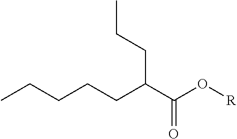

where R is an alkyl radical having 1 to 36 carbon atoms, where R can be substituted by up to 3 OH groups, with the exception of 2-propylheptyl 2-propylheptanoate, 2-ethyl-butyl 2-propylheptanoate and 2-butyloctyl 2-propylheptanoate,
(b) at least one emulsifier (b-1) and/or surfactant (b-2) and/or wax component (b-3) and/or polymer (b-4) and/or a further oil body (b-5).

The present invention further provides cosmetic and/or pharmaceutical preparations comprising
(a) at least one ester according to claim 1
(b) at least one emulsifier (b-1) and/or surfactant (b-2) and/or wax component (b-3) and/or polymer (b-4) and/or a further oil body (b-5).

Preference is given to cosmetic and/or pharmaceutical preparations comprising
(a) at least one ester according to claim 1, with the exception of 2-ethylbutyl 2-propyl-heptanoate and 2-butyloctyl 2-propyl-heptanoate,
(b) at least one emulsifier (b-1) and/or surfactant (b-2) and/or wax component (b-3) and/or polymer (b-4) and/or a further oil body (b-5).

Preferably, the preparations according to the invention comprise 0.1 to 80% by weight, in particular 0.5 to 70% by weight, preferably 0.75 to 60% by weight, in particular 1 to 50% by weight, preferably 1-40% by weight, of at least one ester (a).

The invention further provides cosmetic and/or pharmaceutical preparations comprising
a) 0.1-80% by weight, in particular 0.1 to 70% by weight, preferably 0.1 to 60% by weight, in particular 0.1 to 50% by weight, preferably 0.1-40% by weight, of at least one ester of the general formula (I),

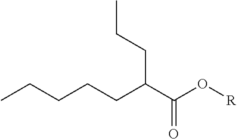

where R is an alkyl radical having 1 to 36 carbon atoms, where R can be substituted by up to 3 OH groups, with the exception of 2-propylheptyl 2-propylheptanoate, preferably with the exception of 2-ethylbutyl 2-propyl-heptanoate and 2-butyloctyl 2-propyl-heptanoate
b) 0.1-20% by weight of emulsifier (b-1) and/or surfactant (b-2) and/or wax component (b-3) and/or polymer (b-4)
b-5) 0.1-40% by weight of further oil bodies and
c) 0-98% by weight of water.

The invention further provides cosmetic and/or pharmaceutical preparations comprising
a) 0.1-80% by weight, in particular 0.1 to 70% by weight, preferably 0.1 to 60% by weight, in particular 0.1 to 50% by weight, preferably 0.1-40% by weight, of at least one ester according to claim 1, preferably of one ester according to claim 1, with the exception of 2-ethylbutyl 2-propylheptanoate and 2-butyloctyl 2-propyl-heptanoate
b) 0.1-20% by weight of emulsifier (b-1) and/or surfactant (b-2) and/or wax component (b-3) and/or polymer (b-4)
b-5) 0.1-40% by weight of further oil bodies and
c) 0-98% by weight of water.

The preparations according to the invention preferably comprise at least 0.1, in particular at least 0.5, in particular at least 0.75, preferably at least 1, preferably at least 5% by weight of one or more esters (a).

All of the % by weight data refer to % by weight based on the cosmetic and/or pharmaceutical preparation.

In one embodiment of the invention, the preparations comprise at least one ester selected from the group consisting of n-hexyl 2-propylheptanoate, n-heptyl 2-propylheptanoate, n-octyl 2-propylheptanoate, n-nonyl 2-propylheptanoate, n-decyl 2-propylheptanoate, n-undecyl 2-propylheptanoate, n-dodecyl 2-propyl-heptanoate or any desired mixtures thereof.

The preparations according to the invention, the compositions according to the invention and the esters according to the invention are suitable to be incorporated as a basis in all cosmetic compositions for body care and body cleaning, such as, for example, body oil, baby oil, body milk, creams, lotions, sprayable emulsions, sunscreen compositions, antiperspirants, liquid and bar soaps etc. They can also be used in surfactant-containing formulations such as, for example, foam and shower baths, hair shampoos and care rinses. They can also be applied as care component on tissues, papers, wipes, nonwoven products, sponges, puffs, plasters and bandages which are used in the field of hygiene and care (wet wipes for baby hygiene and baby care, cleansing wipes, face cleansing wipes, skincare wipes, care wipes containing active ingredients to combat skin ageing, wipes containing sunscreen formulations and insect repellents and also wipes for decorative cosmetics or for aftersun treatment, toilet wet wipes, antiperspirant wipes, diapers, pocket wipes, wet wipes, hygiene products, self-tanning wipes). They can inter alia also be used in preparations for hair care, hair cleansing or hair coloring.

Depending on the intended application, the cosmetic formulations comprise a series of further auxiliaries and additives, such as, for example, surfactants, further oil bodies, emulsifiers, pearlescent waxes, consistency regulators, thickeners, superfatting agents, stabilizers, polymers, fats, waxes, lecithins, phospholipids, biogenic active ingredients, UV sun protection factors, antioxidants, deodorants, antiperspirants, antidandruff agents, film formers, swelling agents, insect repellents, self-tanning agents, tyrosinase inhibitors (depigmentation agents), hydrotropes, solubilizers, preservatives, perfume oils, dyes etc., which are listed below by way of example.

### Emulsifier b-1

In one embodiment of the invention, the preparations according to the invention comprise at least one emulsifier. The compositions according to the invention comprise the emulsifier(s) in an amount of from 0 to 40% by weight, preferably 0.1 to 20% by weight, preferably 0.1 to 15% by weight and in particular 0.1 to 10% by weight, based on the total weight of the composition.

In one embodiment of the invention, the preparation according to the invention comprises more than one emulsifier. The person skilled in the art uses customary emulsifier systems (such as e.g. emulsifier and coemulsifier) depending on the other components.

### Nonionic Emulsifiers

The group of nonionic emulsifiers includes, for example:
(1) addition products of from 2 to 50 mol of ethylene oxide and/or 1 to 20 mol of propylene oxide onto linear fatty alcohols having 8 to 40 carbon atoms, onto fatty acids having 12 to 40 carbon atoms and onto alkylphenols having 8 to 15 carbon atoms in the alkyl group.
(2) C₁₂-C₁₈-Fatty acid mono- and diesters of addition products of from 1 to 50 mol of ethylene oxide onto glycerol.
(3) Sorbitan mono- and diesters of saturated and unsaturated fatty acids having 6 to 22 carbon atoms and ethylene oxide addition products thereof.
(4) Alkyl mono- and oligoglycosides having 8 to 22 carbon atoms in the alkyl radical and ethoxylated analogs thereof.
(5) Addition products of from 7 to 60 mol of ethylene oxide onto castor oil and/or hydrogenated castor oil.
(6) Polyol esters and in particular polyglycerol esters, such as, for example, polyol poly-12-hydroxystearates, polyglycerol polyricinoleate, polyglycerol diisostearate or polyglycerol dimerate. Likewise suitable are mixtures of compounds from two or more of these substance classes.
(7) Addition products of from 2 to 15 mol of ethylene oxide onto castor oil and/or hydrogenated castor oil.
(8) Partial esters based on linear, branched, unsaturated or saturated C₆-C₂₂-fatty acids, ricinoleic acid and 12-hydroxystearic acid and polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose), or mixed esters such as e.g. glyceryl stearate citrate and glyceryl stearate lactate.
(9) Polysiloxane-polyalkyl-polyether copolymers and corresponding derivatives.
(10) Mixed esters of pentaerythritol, fatty acids, citric acid and fatty alcohol and/or mixed esters of fatty acids having 6 to 22 carbon atoms, methylglucose and polyols, preferably glycerol or polyglycerol.

The addition products of ethylene oxide and/or of propylene oxide onto fatty alcohols, fatty acids, alkylphenols, glycerol mono- and diesters and sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. Depending on the degree of ethoxylation, these are W/O or O/W emulsifiers. C_{12/18}-Fatty acid mono- and diesters of addition products of ethylene oxide onto glycerol are known as refatting agents for cosmetic preparations.

Emulsifiers that are mild and particularly suitable according to the invention are polyol poly-12-hydroxystearates and mixtures thereof, which are sold, for example, under the names “Dehymuls® PGPH” (W/O emulsifier) or “Eumulgin® VL 75” (mixture with coco glucosides in the weight ratio 1:1, O/W emulsifier) or Dehymuls® SBL (W/O emulsifier) by Cognis Deutschland GmbH. In this connection, reference may be made in particular to the European patent EP 766 661 B1. The polyol component of these emulsifiers can be derived from substances which have at least two, preferably 3 to 12 and in particular 3 to 8, hydroxy groups and 2 to 12 carbon atoms.

Suitable lipophilic W/O emulsifiers are in principle emulsifiers with an HLB value of from 1 to 8 which are summarized in numerous tables and are known to the person skilled in the art. Some of these emulsifiers are listed, for example, in Kirk-Othmer, “Encyclopedia of Chemical Technology”, 3rd edition, 1979, volume 8, page 913. For ethoxylated products, the HLB value can also be calculated according to the following formula: HLB=(100−L):5, where L is the weight fraction of the lipophilic groups, i.e. of the fatty alkyl or fatty acyl groups, in percent by weight in the ethylene oxide adducts.

From the group of W/O emulsifiers, partial esters of polyols, in particular of C₄-C₆-polyols, such as, for example, partial esters of pentaerythritol or sugar esters, are particularly advantageous, e.g. sucrose distearate, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan mono-ricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical-grade mixtures thereof. Also suitable as emulsifiers are addition products of from 1 to 30, preferably 5 to 10, mol of ethylene oxide onto the specified sorbitan esters.

Depending on the formulation, it may be advantageous to additionally use at least one emulsifier from the group of nonionic O/W emulsifiers (HLB value: 8-18) and/or solubilizers. These are, for example, the ethylene oxide adducts already mentioned in the introduction having a correspondingly high degree of ethoxylation, e.g. 10-20 ethylene oxide units for O/W emulsifiers and 20-40 ethylene oxide units for so-called solubilizers. O/W emulsifiers that are particularly advantageous according to the invention are Ceteareth-12 and PEG-20 stearate. Suitable solubilizers are preferably Eumulgin® HRE 40 (INCI: PEG-40 Hydrogenated Castor Oil), Eumulgin® HRE 60 (INCI: PEG-60 Hydrogenated Castor Oil), Eumulgin® L (INCI: PPG-1-PEG-9 lauryl glycol ether), and Eumulgin® SML 20 (INCI: Polysorbate-20).

Nonionic emulsifiers from the group of alkyl oligoglycosides are particularly skin-friendly and therefore preferably suitable as O/W emulsifiers. C₈-C₂₂-Alkyl mono- and oligoglycosides, their preparation and their use are known from the prior art. Their preparation takes place in particular by reacting glucose or oligosaccharides with primary alcohols having 8 to 22 carbon atoms. As regards the glycoside radical, both monoglycosides, in which one cyclic sugar radical is glycosidically bonded to the fatty alcohol, and also oligomeric glycosides with a degree of oligomerization up to preferably about 8 are suitable. The degree of oligomerization here is a statistical average value based on a homolog distribution customary for such technical-grade products. Products which are available under the name Plantacare® comprise a glucosidically bonded C₈-C₁₆-alkyl group onto an oligoglucoside radical whose average degree of oligomerization is 1 to 2. The acylglucamides derived from glucamine are also suitable as nonionic emulsifiers. According to the invention, preference is given to a product which is sold under the name Emulgade® PL 68/50 by Cognis Deutschland GmbH and is a 1:1 mixture of alkyl polyglucosides and fatty alcohols. According to the invention, it is also advantageously possible to use a mixture of laurylglucoside, polyglyceryl-2 dipolyhydroxystearate, glycerol and water, which is commercially available under the name Eumulgin® VL 75.

Suitable emulsifiers are also substances such as lecithins and phospholipids. Examples of natural lecithins which may be mentioned are the cephalins, which are also referred to as phosphatidic acids and are derivatives of 1,2-diacyl-sn-glycerol-3-phosphoric acids. By contrast, phospholipids are usually understood as meaning mono- and preferably diesters of phosphoric acid with glycerol (glycerol phosphates), which are generally included among the fats. In addition, sphingosines and/or sphingolipids are also suitable.

### Surfactants b-2

In one embodiment of the invention, the preparations according to the invention comprise at least one surfactant. Surface-active substances which may be present are anionic, nonionic, cationic and/or amphoteric or zwitterionic surfactants. In surfactant-containing cosmetic preparations, such as, for example, shower gels, foam baths, shampoos etc., at least one anionic surfactant is preferably present.

The compositions according to the invention comprise the surfactant(s) in an amount of from 0 to 40% by weight, preferably 0 to 20% by weight, preferably 0.1 to 15% by weight and in particular 0.1 to 10% by weight, based on the total weight of the composition.

Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partially oxidized alk(en)yl oligoglycosides and glucoronic acid derivatives, fatty acid N-alkylglucamides, protein hydrolyzates (in particular wheat-based vegetable products), polyol fatty acid esters, sugar esters, sorbitan esters, polysorbates and amine oxides. If the nonionic surfactants comprise polyglycol ether chains, these can have a conventional homolog distribution, but preferably have a narrowed homolog distribution.

Zwitterionic surfactants is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazoline having in each case 8 to 18 carbon atoms in the alkyl or acyl group, and cocoacylaminoethylhydroxyethyl-carboxymethyl glycinate. A preferred zwitterionic surfactant is the fatty acid amide derivative known under the INCI name Cocamidopropyl Betaine.

Likewise suitable, particularly as cosurfactants, are ampholytic surfactants. Ampholytic surfactants are understood as meaning those surface-active compounds which, apart from a C₈-C₁₈-alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkylimino-dipropionic acids, N-hydroxyethyl-N-alkylamidopropyl-glycines, N-alkyltaurines, N-alkylsarcosines, 2-alkyl-aminopropionic acids and alkylaminoacetic acids having in each case about 8 to 18 carbon atoms in the alkyl group. Particularly preferred ampholytic surfactants are N-cocoalkylaminopropionate, cocoacylaminoethyl-aminopropionate and C₁₂₋₁₈-acylsarcosine.

Anionic surfactants are characterized by a water-solubilizing, anionic group such as, for example, a carboxylate, sulfate, sulfonate or phosphate group and a lipophilic radical. Skin-compatible anionic surfactants are known to the person skilled in the art in a large number from relevant handbooks and are commercially available. These are in particular alkyl sulfates in the form of their alkali metal, ammonium or alkanolammonium salts, alkyl ether sulfates, alkyl ether carboxylates, acyl isethionates, acyl sarcosinates, acyltaurines with linear alkyl or acyl groups having 12 to 18 carbon atoms, and sulfosuccinates and acyl glutamates in the form of their alkali metal or ammonium salts.

Cationic surfactants which can be used are in particular quaternary ammonium compounds. Preference is given to ammonium halides, in particular chlorides and bromides, such as alkyltrimethylammonium chlorides, dialkyldimethylammonium chlorides and trialkylmethyl-ammonium chlorides, e.g. cetyltrimethylammonium chloride, stearyltrimethylammonium chloride, distearyl-dimethylammonium chloride, lauryldimethylammonium chloride, lauryldimethylbenzylammonium chloride and tricetylmethylammonium chloride. Furthermore, the very readily biodegradable quaternary ester compounds, such as, for example, the dialkylammonium methosulfates and methylhydroxyalkyldialkoyloxyalkylammonium methosulfates sold under the trade name Stepantex® and the corresponding products of the Dehyquart® series, can be used as cationic surfactants. The term “ester quats” is generally understood as meaning quaternized fatty acid triethanolamine ester salts. They can impart a particular soft feel to the compositions according to the invention. These are known substances which are prepared by the relevant methods of organic chemistry. Further cationic surfactants that can be used according to the invention are the quaternized protein hydrolyzates.

### Wax Component b-3

In one embodiment of the invention, the preparations according to the invention comprise at least one wax component. The compositions according to the invention comprise the wax component(s) in an amount of from 0 to 40% by weight, in particular from 0 to 20% by weight, preferably 0.1 to 15% by weight and in particular 0.1 to 10% by weight, based on the total weight of the composition.

The term wax is generally understood as meaning all natural or synthetically obtained substances and substance mixtures with the following properties: they are of solid to brittly hard consistency, coarse to finely crystalline, transparent to opaque and melt above 30° C. without decomposition. Even a little above the melting point, they are of low viscosity and not thread-drawing and exhibit a highly temperature-dependent consistency and solubility. According to the invention, it is possible to use a wax component or a mixture of wax components which melt at 30° C. or above.

According to the invention, waxes which can be used are also fats and fat-like substances with wax-like consistency provided they have the required melting point. These include, inter alia, fats (triglycerides), mono- and diglycerides, natural and synthetic waxes, fatty alcohols and wax alcohols, fatty acids, esters of fatty alcohols and fatty acids, and fatty acid amides or any desired mixtures of these substances.

Fats are understood as meaning triacylglycerols, i.e. the triple esters of fatty acids with glycerol. Preferably, they comprise saturated, unbranched and unsubstituted fatty acid radicals. These may be mixed esters, i.e. triple esters of glycerol with various fatty acids. So-called hydrogenated fats and oils obtained by partial hydrogenation can be used according to the invention and are particularly suitable as consistency regulators. Vegetable hydrogenated fats and oils are preferred, e.g. hydrogenated castor oil, peanut oil, soybean oil, colza oil, rapeseed oil, cottonseed oil, soybean oil, sunflower oil, palm oil, palm kernel oil, linseed oil, almond oil, corn oil, olive oil, sesame oil, cocoa butter and coconut fat.

Inter alia, the triple esters of glycerol with C12-C60-fatty acids and in particular C12-C36-fatty acids are suitable. These include hydrogenated castor oil, a triple ester of glycerol and a hydroxystearic acid, which is commercially available, for example, under the name Cutina HR. Glycerol tristearate, glycerol tribehenate (e.g. Syncrowax HRC), glycerol tripalmitate or the triglyceride mixtures known under the name Syncrowax HGLC are likewise suitable, with the proviso that the melting point of the wax component or of the mixture is 30° C. or above.

According to the invention, wax components which can be used are in particular mono- and diglycerides and mixtures of these partial glycerides. Glyceride mixtures that can be used according to the invention include the products Novata AB and Novata B (mixture of C12-C18-mono-, di- and triglycerides) and Cutina MD or Cutina GMS (glyceryl stearate) marketed by Cognis Deutschland GmbH & Co. KG.

Fatty alcohols which can be used according to the invention as wax component include the C12-C50-fatty alcohols. The fatty alcohols can be obtained from natural fats, oils and waxes, such as, for example, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. According to the invention, saturated, unbranched fatty alcohols are preferred. However, unsaturated, branched or unbranched fatty alcohols can also be used according to the invention as wax component provided they have the required melting point. According to the invention, it is also possible to use fatty alcohol cuts as are produced during the reduction of naturally occurring fats and oils, such as, for example, bovine tallow, peanut oil, colza oil, cottonseed oil, soybean oil, sunflower oil, palm kernel oil, linseed oil, castor oil, corn oil, rapeseed oil, sesame oil, cocoa butter and coconut fat. However, it is also possible to use synthetic alcohols, e.g. the linear, even-numbered fatty alcohols of the Ziegler synthesis (alfols) or the partially branched alcohols from the oxo synthesis (dobanols). According to the invention, C14-C22-fatty alcohols, which are marketed, for example, by Cognis Deutschland GmbH under the name Lanette 16 (C16-alcohol), Lanette 14 (C14-alcohol), Lanette O (C16/C18-alcohol) and Lanette 22 (C18/C22-alcohol), are particularly preferably suitable. Fatty alcohols give the compositions a drier skin feel than triglycerides and are therefore preferred over the latter.

Wax components which can be used are also C14-C40-fatty acids or mixtures thereof. These include, for example, myristic acid, pentadecanoic acid, palmitic acid, margaric acid, stearic acid, nonadecanoic acid, arachic acid, behenic acid, lignoceric acid, cerotic acid, melissic acid, erucic acid and elaeostearic acid, and substituted fatty acids, such as, for example, 12-hydroxystearic acid, and the amides or monoethanolamides of the fatty acids, this list being exemplary and nonlimiting in character.

According to the invention, it is possible to use, for example, natural vegetable waxes, such as candelilla wax, carnauba wax, japan wax, esparto grass wax, cork wax, guaruma wax, rice germ oil wax, sugar cane wax, ouricury wax, montan wax, sunflower wax, fruit waxes such as orange waxes, lemon waxes, grapefruit wax, bayberry wax, and animal waxes, such as, for example, beeswax, shellac wax, spermaceti, wool wax and uropygial fat. Within the context of the invention, it may be advantageous to use hydrogenated or hardened waxes. Natural waxes that can be used according to the invention also include the mineral waxes, such as, for example, ceresin and ozokerite or the petrochemical waxes, such as, for example, petrolatum, paraffin waxes and microwaxes. Wax components which can be used are also chemically modified waxes, in particular the hard waxes, such as, for example, montan ester waxes, sasol waxes and hydrogenated jojoba waxes. Synthetic waxes which can be used according to the invention include, for example, wax-like polyalkylene waxes and polyethylene glycol waxes. Vegetable waxes are preferred according to the invention.

The wax component can likewise be selected from the group of wax esters of saturated and/or unsaturated, branched and/or unbranched alkane carboxylic acids and saturated and/or unsaturated, branched and/or unbranched alcohols, from the group of esters of aromatic carboxylic acids, dicarboxylic acids, tricarboxylic acids and hydroxycarboxylic acids (e.g. 12-hydroxystearic acid) and saturated and/or unsaturated, branched and/or unbranched alcohols, and also from the group of lactides of long-chain hydroxycarboxylic acids. Examples of such esters are the C16-C40-alkyl stearates, C20-C40-alkyl stearates (e.g. Kesterwachs K82H), C20-C40-dialkyl esters of dimer acids, C18-C38-alkylhydroxystearoyl stearates or C20-C40-alkyl erucates. C30-C50-Alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate can also be used.

### Polymers b-4

In one embodiment of the invention, the preparations according to the invention comprise at least one polymer. The compositions according to the invention comprise the polymer(s) in an amount of from 0 to 20% by weight, preferably 0.1 to 15% by weight and in particular 0.1 to 10% by weight, based on the total weight of the composition.

Suitable cationic polymers are, for example, cationic cellulose derivatives, such as, for example, a quaternized hydroxyethylcellulose which is available under the name Polymer JR 400® from Amerchol, cationic starch, copolymers of diallylammonium salts and acrylamides, quaternized vinylpyrrolidone/vinyl-imidazole polymers, such as, for example, Luviquat (BASF), condensation products of polyglycols and amines, quaternized collagen polypeptides, such as, for example, lauryldimonium hydroxypropyl hydrolyzed collagen (Lamequat®L/Grunau), quaternized wheat polypeptides, polyethyleneimine, cationic silicone polymers, such as, for example, amidomethicones, copolymers of adipic acid and dimethyl-aminohydroxypropyldiethylenetriamine (Cartaretine®/Sandoz), copolymers of acrylic acid with dimethyldiallylammonium chloride (Merquat® 550/Chemviron), polyaminopolyamides, cationic chitin derivatives, such as, for example, quaternized chitosan, optionally in microcrystalline distribution, condensation products of dihaloalkylene, such as, for example, dibromobutane with bisdialkylamines, such as, for example, bisdimethylamino-1,3-propane, cationic guar gum, such as, for example, Jaguar® CBS, Jaguar® C-17, Jaguar® C-16 from Celanese, quaternized ammonium salt polymers, such as, for example, Mirapol® A-15, Mirapol® AD-1, Mirapol® AZ-1 from Miranol.

Suitable anionic, zwitterionic, amphoteric and nonionic polymers are, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyltrimethylammonium chloride/acrylate copolymers, octylacrylamide/methyl methacrylate/tert-butylaminoethyl methacrylate/2-hydroxypropyl methacylate copolymers, polyvinylpyrrolidone, vinylpyrrolidone/vinyl acetate copolymers, vinylpyrrolidone/dimethylaminoethyl methacrylate/vinylcaprolactam terpolymers, and optionally derivatized cellulose ethers and silicones.

Suitable polymers are likewise polysaccharides, in particular xanthan gum, guar-guar, agar-agar, alginates and tyloses.

### Further Oil Bodies b-5

Body care compositions such as creams, body oils, lotions and milks usually comprise a series of further oil bodies and emollients which contribute to further optimizing the sensory properties. The oil bodies (esters according to the invention plus further oil bodies) are usually present in a total amount of 0.1 -80% by weight, in particular 0.5 to 70% by weight, preferably 1 to 60% by weight, in particular 1 to 50% by weight, in particular 1 to 40% by weight, preferably 5-25% by weight and in particular 5-15% by weight. The further oil bodies are usually present in an amount of from 0.1 to 40% by weight.

Suitable further oil bodies are, for example, Guerbet alcohols based on fatty alcohols having 6 to 18, preferably 8 to 10, carbon atoms, and also further additional esters such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Likewise suitable are esters of C₁₈-C₃₈-alkylhydroxycarboxylic acids with linear or branched C₆-C₂₂-fatty alcohols, in particular dioctyl malate, esters of linear and/or branched fatty acids with polyhydric alcohols (such as, for example, propylene glycol, dimerdiol or trimertriol), triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonate (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols and hydrocarbons or mixtures thereof (Cetiol® DD).

### Further Ingredients

Suitable thickeners are, for example, Aerosil grades (hydrophilic silicas), carboxymethylcellulose and hydroxyethyl- and hydroxypropylcellulose, polyvinyl alcohol, polyvinylpyrrolidone and bentonites such as, for example, Bentone® Gel VS-5PC (Rheox).

UV sun protection factors are to be understood, for example, as meaning organic substances (photoprotective filters) which are present in crystalline or liquid form at room temperature and which are able to absorb ultraviolet rays and release the absorbed energy again in the form of longer-wave radiation, e.g. heat. UV-B filters may be oil-soluble or water-soluble. Suitable typical UV-A filters are in particular derivatives of benzoylmethane. The UV-A and UV-B filters may of course also be used in mixtures, e.g. combinations of the derivatives of benzoylmethane, e.g. 4-tert-butyl-4′-methoxydibenzoylmethane (Parsol⁵ 1789) and 2-ethylhexyl 2-cyano-3,3-phenylcinnamate (octocrylene), and esters of cinnamic acid, preferably 2-ethylhexyl 4-methoxy-cinnamate and/or propyl 4-methoxycinnamate and/or isoamyl 4-methoxycinnamate. Such combinations are often combined with water-soluble filters such as, for example, 2-phenylbenzimidazole-5-sulfonic acid and its alkali metal, alkaline earth metal, ammonium, alkylammonium, alkanolammonium and glucammonium salts.

Besides the specified soluble substances, insoluble photoprotective pigments, namely finely disperse metal oxides, are also suitable. Examples of suitable metal oxides are in particular zinc oxide and titanium dioxide. Besides the two aforementioned groups of primary photoprotective substances, it is also possible to use secondary photoprotective agents of the antioxidant type which interrupt the photochemical reaction chain which is triggered when UV radiation penetrates into the skin.

Biogenic active ingredients are to be understood as meaning, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, such as, for example, prune extract, bambara nut extract and vitamin complexes.

Deodorizing active ingredients antiperspirants counteract, mask or eliminate body odors. Body odors are formed through the action of skin bacteria on apocrine perspiration, during which unpleasant-smelling degradation products are formed. Accordingly, suitable deodorizing active ingredients are, inter alia, antibacterial agents, enzyme inhibitors, odor absorbers or odor maskers.

Suitable insect repellents are, for example, N,N-diethyl-m-toluamide, 1,2-pentanediol or ethyl 3-(N-n-butyl-N-acetylamino)propionate, which is sold under the name Insect Repellent® 3535 by Merck KGaA, and also butylacetylaminopropionates.

A suitable self-tanning agent is dihydroxyacetone. Suitable tyrosine inhibitors, which prevent the formation of melanin and are used in depigmentation compositions, are, for example, arbutin, ferulic acid, kojic acid, coumaric acid and ascorbic acid (vitamin C).

Suitable preservatives are, for example, phenoxyethanol, formaldehyde solution, parabens, pentanediols or sorbic acid, and also the silver complexes known under the name Surfacine® and the further substance classes listed in Appendix 6, Part A and B of the Cosmetics Ordinance.

Perfume oils which may be mentioned are mixtures of natural and synthetic fragrances. Natural fragrances are extracts from flowers, stems and leaves, fruits, fruit peels, roots, woods, herbs and grasses, needles and branches, resins and balsams. Also suitable are animal raw materials, such as, for example, civet and castoreum, and also synthetic fragrance compounds of the ester, ether, aldehyde, ketone, alcohol and hydrocarbon types.

Suitable pearlescent waxes, in particular for use in surface-active formulations, are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have in total at least 24 carbon atoms, specifically laurone and distearyl ether; fatty acids such as stearic acid, hydroxystearic acid or behenic acid, ring-opening products of olefin epoxides having 12 to 22 carbon atoms with fatty alcohols having to 22 carbon atoms and/or polyols having 2 to 15 carbon atoms and 2 to 10 hydroxyl groups, and mixtures thereof.

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter simultaneously serving as foam stabilizers.

Stabilizers which can be used are metal salts of fatty acids, such as, for example, magnesium, aluminum and/or zinc stearate and/or ricinoleate.

To improve the flow behavior, hydrotropes, such as, for example, ethanol, isopropyl alcohol, or polyols, can also be used. Polyols which are suitable here preferably have 2 to 15 carbon atoms and at least two hydroxyl groups. The polyols can also contain further functional groups, in particular amino groups, and/or be modified with nitrogen.

The preparations according to the invention, the compositions according to the invention and the esters according to the invention are suitable in particular in cosmetic and/or pharmaceutical preparations for the wetting or impregnation or coating of utility wipes and hygiene wipes which are used for body cleaning and/or for body care.

Utility wipes and hygiene wipes which may be mentioned by way of example are: tissues, papers, wipes, nonwoven products, sponges, puffs, plasters and bandages which are used in the field of hygiene and care. These may be wet wipes for baby hygiene and baby care, cleansing wipes, face cleansing wipes, skincare wipes, care wipes containing active ingredients to combat skin ageing, wipes containing sunscreen formulations and insect repellents, and also wipes for decorative cosmetics or for aftersun treatment, toilet wet wipes, antiperspirant wipes, diapers, pocket wipes, wet wipes, hygiene products and self-tanning wipes.

### EXAMPLE

64.8 g of 2-propylheptanoic acid (AN=111.1), 16.8 g of octanol and 0.3 g of Sn oxalate were heated together on a water separator at 220° C. for 6 h. The resulting crude product (AN=2.4) was distilled using a 250 mm Vigreux column in an oil pump vacuum. 42 g of purified n-octyl 2-propylheptanoate are produced as a yellowish oil at 0.04 mbar and 114-117° C.

1. An isolated ester of general formula (I), 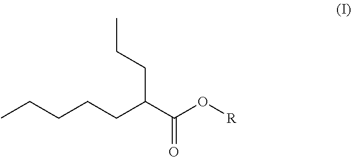
where R is an alkyl radical group having 6 to 12 carbon atoms, R optionally substituted by up to 3 OH groups, excluding the ester 2-propylheptyl 2-propylheptanoate, .

2. (canceled)

3. The ester of claim 1, selected from the group consisting, selected from the group consisting of n-hexyl 2-propylheptanoate, n-heptyl 2-propylheptanoate, n-octyl 2-propylheptanoate, n-nonyl 2-propylheptanoate, n-decyl 2-propylheptanoate, n-undecyl 2-propylheptanoate, n-dodecyl 2-propylheptanoate.

4-6. (canceled)

7. A cosmetic and/or pharmaceutical composition comprising
(a) at least one ester of general formula (I), 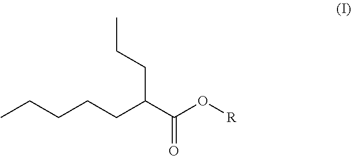
where R is an alkyl group having 1 to 36 carbon atoms, optionally substituted by up to 3 OH groups, excluding the ester 2-propylheptyl 2-propylheptanoate, and
(b) at least one pharmaceutically and/or cosmetically acceptable emulsifier (b-1), and/or surfactant (b-2), and/or wax component (b-3), and/or polymer (b-4), and/or a further oil body (b-5),
wherein the compositions is free from esters of general formula (I) having branched alkyl groups as the R group.

8. The composition as claimed in of claim 7, comprising 0.1-80% by weight, of at least one ester (a) according to formula (I).

9. The composition of claim 7, comprising, as component (a), at least one ester selected from the group consisting of n-hexyl 2-propylheptanoate, n-heptyl 2-propylheptanoate, n-octyl 2-propylheptanoate, n-nonyl 2-propylheptanoate, n-decyl 2-propylheptanoate, n-undecyl 2-propylheptanoate, n-dodecyl 2-propylheptanoate and combinations thereof.

10. The composition of claim 8, comprising 0.1-40% by weight, of at least one ester (a) according to formula (I).
